(19) United States
(12) Patent Application Publication
(10) Pub. No.: US 2025/0114101 A1
(43) Pub. Date: Apr. 10, 2025

(54) Surgical Driving System For A Surgical Device

(71) Applicant: MAKO Surgical Corp., Weston, FL (US)

(72) Inventors: Jonathan P. Boyer, Galesburg, MI (US); Richard Wayne Mitchell, Kalamazoo, MI (US)

(73) Assignee: MAKO Surgical Corp., Weston, FL (US)

(21) Appl. No.: 18/904,347

(22) Filed: Oct. 2, 2024

Related U.S. Application Data
(60) Provisional application No. 63/542,370, filed on Oct. 4, 2023.

(51) Int. Cl. A61B 17/16; A61B 17/00

(52) U.S. Cl. CPC A61B 17/1631; A61B 17/1617; A61B 17/1628; A61B 2017/00486

(57) ABSTRACT

A surgical driving system includes a driver and a cutting accessory. The driver includes a body driven by a motor. First and second arms project from the body, each arm having its own alignment tip. The arms define a channel between each other. The alignment tips are offset. Each tip includes two alignment surfaces facing away from each other. The cutting accessory includes a distal portion having a cutting tip and being coupled to a tool shaft. A proximal portion of the cutting accessory is coupled to the tool shaft and received within the channel. The proximal portion has a drive section with a flat to engage the arms of the driver. The proximal portion also has an alignment section separate from and proximal to the drive section to engage the alignment tips of the driver to orient the drive section relative to the arms.

### CROSS-REFERENCE TO RELATED APPLICATIONS

The subject application claims priority to and all the benefits of U.S. Provisional Patent App. No. 63/542,370, filed Oct. 4, 2023, the entire contents of which are hereby incorporated by reference.

### BACKGROUND

Robotic systems are commonly used to perform surgical procedures and typically include a robot comprising a robotic arm and an end effector coupled to an end of the robotic arm and presenting a tool. The end effector includes a handle for manipulating the position of the tool.

In many conventional systems, the end effectors utilize a driver for transferring torque to a cutting accessory to rotate the cutting accessory. Cutting accessories are often removably coupled to the driver. In order for the driver to transmit torque to the cutting accessory, the cutting accessory must be correctly oriented to the driver so that the cutting accessory is engaged with the driver. It is often difficult to observe the correct orientation of the cutting accessory needed to establish engagement between the cutting accessory and the driver to permit torque transmission between the cutting accessory and the driver.

As such, there is a need in the art for drivers and cutting accessories that address at least the aforementioned problems.

### SUMMARY

According to a first aspect, a cutting accessory for a surgical cutting tool is provided. The cutting accessory includes a tool shaft comprising an axis and being rotatable about the axis. The cutting accessory also includes a distal portion coupled to the tool shaft and having a cutting tip. The cutting accessory further includes a proximal portion coupled to the tool shaft and configured to engage a driver of the surgical cutting tool. The proximal portion comprises a drive section having one or more flats configured to engage drive surfaces of the driver to receive torque from the driver. The proximal portion also includes an alignment section distinct from and proximal to the drive section. The alignment section is configured to orient the drive section to the drive surfaces of the driver. The alignment section includes a proximal end comprising at least one edge. The alignment section also includes an alignment surface extending distally from the at least one edge of the proximal end toward the drive section. The alignment surface is twisted relative to the axis between the at least one edge of the proximal end and the drive section.

According to a second aspect, a driver of a surgical tool for engaging a cutting accessory is provided. The driver includes a body having a proximal portion configured to receive torque from a motor and rotate about an axis. The driver also includes a first arm projecting distally from the body. The first arm has a first alignment tip. The driver further includes a second arm projecting distally from the body and being spaced from the first arm. The second arm has a second alignment tip. The first and second arms collectively define a channel between the first and second arms for at least partially receiving the cutting accessory. Each of the alignment tips include two alignment surfaces facing away from each other. The two alignment surfaces are arranged for selectively imparting rotation of the cutting accessory during engagement with the cutting accessory. The second alignment tip is offset from the first alignment tip such that the first alignment tip is located on one side of a reference plane that bisects body along the axis and the second alignment tip is located on an opposing side of the reference plane.

According to a third aspect, a surgical driving system for transmitting torque received from a motor is provided. The surgical driving system further comprises a driver configured to receive torque from the motor. The driver comprises a body having a proximal portion configured to receive torque from the motor and rotate about an axis. The driver also comprises a first arm projecting distally from the body. The first arm has a first alignment tip. The driver further comprises a second arm projecting distally from the body being spaced from the first arm. The second arm has a second alignment tip. The first and second arms collectively define a channel between the first and second arms. Each of the alignment tips include two alignment surfaces facing away from each other. The second alignment tip is offset from the first alignment tip such that the first alignment tip is located on one side of a reference plane that bisects the body along an axis and the second alignment tip is located on an opposing side of the reference plane. The surgical driving system also includes a cutting accessory configured to be coupled to the driver. The cutting accessory comprises a tool shaft comprising an axis and being rotatable about the axis. The cutting accessory also comprises a distal portion coupled to the tool shaft and having a cutting tip. The cutting accessory also comprises a proximal portion coupled to the tool shaft and configured to be received within the channel to engage the driver. The proximal portion includes a drive section having one or more flats configured to engage the first and second arms of the driver to receive torque from the driver. The proximal portion also includes an alignment section distinct from and proximal to the drive section. The alignment section is configured to engage the alignment tips of the driver to orient the drive section relative to the arms.

According to a fourth aspect, another surgical driving system for transmitting torque received from a motor is provided. The surgical driving system also comprises a driver configured to receive torque from the motor. The driver comprises a body and an arm projecting distally from the body. The arm includes an alignment tip and a planar drive surface. The alignment tip includes a first helically contoured alignment surface. The surgical driving system also comprises a cutting accessory configured to be coupled to the driver. The cutting accessory comprises a tool shaft, a distal portion including a cutting tip, and a proximal portion comprising a planar driven surface. The proximal portion further includes a second helically contoured alignment surface that is configured to engage the first helically contoured alignment surface to rotatably align the drive and driven surfaces.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a robotic system for manipulating a target tissue of a patient with a tool, according to one example.

FIG. 2 is a perspective view of an end effector for use with the robotic system.

FIG. 3 is a sectional view of the end effector.

FIG. 4 is an exploded view of a cutting accessory and a drive assembly including a driver and a drive member.

FIG. 5 is a sectional view of the cutting accessory and drive assembly of FIG. 4 taken along a first reference plane.

FIG. 6 is another sectional view of the cutting accessory and the drive assembly of FIG. 4 taken along a second referent plane perpendicular to the first reference plane.

FIG. 7 is a sectional view of the drive member.

FIG. 8 is a side elevation view of the driver.

FIG. 9 is another side elevation view of the driver.

FIG. 10 is a front elevation view of a distal end of the drive assembly.

FIG. 11 is a section view of the drive assembly.

FIG. 12 is a perspective view of a proximal portion of the cutting accessory.

FIG. 13 is a back elevation view of a proximal end of the cutting accessory.

FIG. 14 is a side elevation view of the proximal portion of the cutting accessory.

FIG. 15 is another side elevation view of the proximal portion of the cutting accessory.

### DETAILED DESCRIPTION

### I. Robotic System Overview

Referring to the Figures, wherein like numerals indicate like or corresponding parts throughout the several views, a system 10 (hereinafter “system”) is shown throughout.

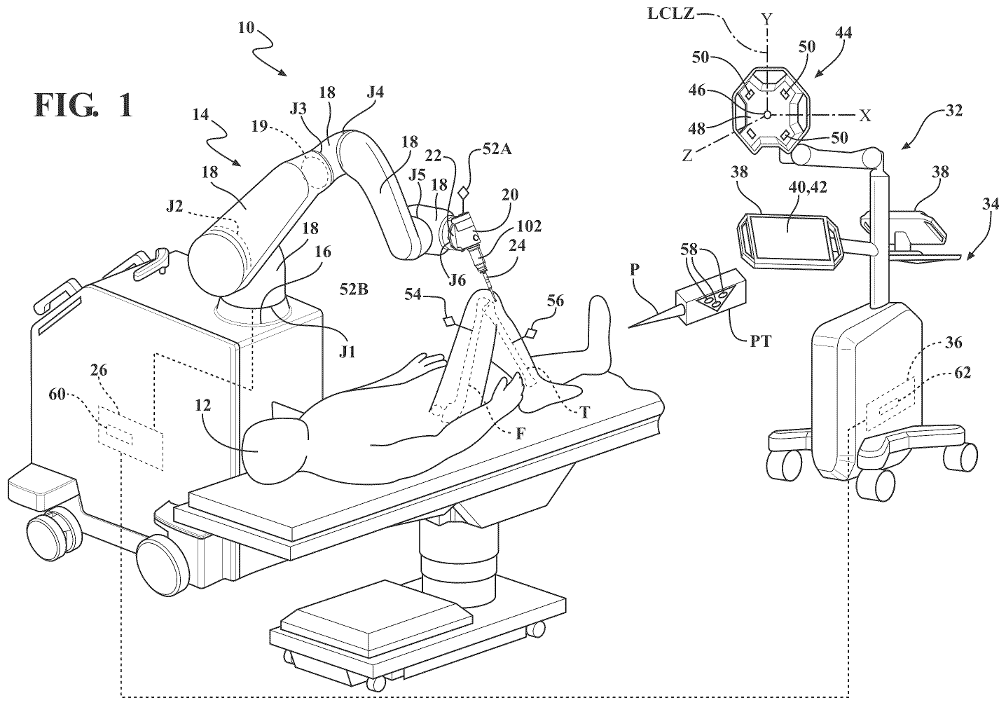

As shown in FIG. 1, the system 10 may treat an anatomy (surgical site) of a patient 12, such as bone or soft tissue. In FIG. 1, the patient 12 is undergoing a surgical procedure. The anatomy in FIG. 1 includes a femur (F) and a tibia (T) of the patient 12. The surgical procedure may involve tissue removal or treatment. Treatment may include cutting, coagulating, lesioning the tissue, treatment in place of tissue, or the like. In some examples, the surgical procedure involves partial or total knee or hip replacement surgery. In one example, the system 10 is designed to cut away material to be replaced by surgical implants, such as hip and knee implants, including unicompartmental, bicompartmental, multicompartmental, or total knee implants. Some of these types of implants are shown in U.S. Pat. No. 9,937,058, entitled, “Prosthetic Implant and Method of Implantation,” the disclosure of which is hereby incorporated by reference. The system 10 disclosed herein may be used to perform other procedures, surgical or non-surgical, or may be used in industrial applications or other applications where robotic systems are utilized.

The system 10 may include a robotic manipulator 14. The robotic manipulator 14 has a base 16 and plurality of links 18. A manipulator cart 17 supports the robotic manipulator 14 such that the robotic manipulator 14 is fixed to the manipulator cart 17. The links 18 collectively form one or more arms of the robotic manipulator 14. The robotic manipulator 14 may have a serial arm configuration (as shown in FIG. 1) or a parallel arm configuration. In other examples, more than one robotic manipulator 14 may be utilized in a multiple arm configuration. The robotic manipulator 14 may comprise a plurality of (prismatic and/or rotating) joints (J) and a plurality of motor and/or joint encoders 19 located at the joints (J) for determining position data of the joints (J). For simplicity, only one joint encoder 19 is illustrated in FIG. 1, although it is to be appreciated that the other joint encoders 19 may be similarly illustrated. The robotic manipulator 14 according to one example has six joints (J1-J6) implementing at least six-degrees of freedom (DOF) for the robotic manipulator 14. However, the robotic manipulator 14 may have any number of degrees of freedom and may have any suitable number of joints (J) and redundant joints (J).

A surgical tool 20 (hereinafter “tool”) couples to the robotic manipulator 14 and is movable relative to the base 16 to interact with the anatomy in certain modes. The tool 20 is or can form part of an end effector 22. The tool 20 may be grasped by the operator. One exemplary arrangement of the robotic manipulator 14 and the tool 20 is described in U.S. Pat. No. 9,119,655, entitled, “Surgical Manipulator Capable of Controlling a Surgical Instrument in Multiple Modes,” the disclosure of which is hereby incorporated by reference. The robotic manipulator 14 and the tool 20 may be arranged in alternative configurations. The tool 20 can be like that shown in U.S. Pat. No. 9,566,121, filed on Mar. 15, 2014, entitled, “End Effector of a Surgical Robotic Manipulator,” hereby incorporated by reference. Another suitable tool 20 can be like that shown in U.S. Patent Publication No. 2022/0087754A1, filed on Sep. 24, 2021, entitled, “Interlocking Collet System For A Surgical Device,” hereby incorporated by reference.

The positioning of the end effector 22 and the tool 20 is defined by the robotic manipulator 14. This positioning may not be ideally suited for the ergonomics of an operator. To that end, the end effector 22 may include a handle 102 that is rotatable about a rotational axis R. The rotatable handle 102 allows the operator to hold the tool 20 in the most comfortable position while the robotic manipulator 14 moves the tool 20 into the necessary position for robotic manipulation. Exemplary arrangements of the handle 102 rotatable about the rotational axis R are described in U.S. Pat. No. 9,566,121, entitled, “End Effector of a Surgical Robotic Manipulator,” and U.S. Patent Application Publication No. 2018/0110572, filed on Oct. 21, 2016, entitled, “Systems and Tools for Use with Surgical Robotic Manipulators,” and the aforementioned U.S. Patent Publication No. 2022/0087754A1, the disclosures of which are hereby incorporated by reference.

The tool 20 includes an energy applicator 24 designed to contact the target site, such as the tissue of the patient 12 at the surgical site. The energy applicator 24 may be a drill, a saw blade, a bur, an ultrasonic vibrating tip, or the like. In some configurations, the energy applicator may comprise the cutting accessory 108.

The system 10 includes a controller 30. The controller 30 includes software and/or hardware for controlling the robotic manipulator 14. The controller 30 directs the motion of the robotic manipulator 14 and controls a state (position and/or orientation) of the tool 20 with respect to a coordinate system of the manipulator 14.

As shown in FIG. 1, the system 10 further includes a navigation system 32. One example of the navigation system 32 is described in U.S. Pat. No. 9,008,757, filed on Sep. 24, 2013, entitled, “Navigation System Including Optical and Non-Optical Sensors,” hereby incorporated by reference. The navigation system 32 is configured to track movement of various objects. Such objects include, for example, the robotic manipulator 14, the tool 20 and the anatomy, e.g., femur F and tibia T. The navigation system 32 tracks these objects to gather state information of each object with respect to a (navigation) localizer coordinate system LCLZ. Coordinates in the localizer coordinate system LCLZ may be transformed to the manipulator coordinate system MNPL, and/or vice-versa, using transformation techniques described herein.

The navigation system 32 includes a cart assembly 34 that houses a navigation computer 36, and/or other types of control units. A navigation interface is in operative communication with the navigation computer 36. The navigation interface includes one or more displays 38. First and second input devices 40, 42 may be used to input information into the navigation computer 36 or otherwise to select/control certain aspects of the navigation computer 36. As shown in FIG. 1, such input devices 40, 42 include interactive touchscreen displays. However, the input devices 40, 42 may include any one or more of a keyboard, a mouse, a microphone (voice-activation), gesture control devices, and the like. The controller 30 may be implemented on any suitable device or devices in the system 10, including, but not limited to, the manipulator computer 26, the navigation computer 36, and any combination thereof.

The navigation system 32 also includes a navigation localizer 44 (hereinafter “localizer”) coupled to the navigation computer 36. In one example, the localizer 44 is an optical localizer and includes a camera unit 46. The camera unit 46 has an outer casing 48 that houses one or more optical sensors 50.

The navigation system 32 includes one or more trackers. In one example, the trackers include a pointer tracker PT, one or more manipulator trackers 52, a first patient tracker 54, and a second patient tracker 56. In the illustrated example of FIG. 1, the manipulator tracker 52 is firmly attached to the tool 20 (i.e., tracker 52A), the first patient tracker 54 is firmly affixed to the femur F of the patient 12, and the second patient tracker 56 is firmly affixed to the tibia T of the patient 12. In this example, the patient trackers 54, 56 are firmly affixed to sections of bone. The pointer tracker PT is firmly affixed to a pointer P used for registering the anatomy to the localizer coordinate system LCLZ. The manipulator tracker 52 may be affixed to any suitable component of the robotic manipulator 14, in addition to, or other than the tool 20, such as the base 16 (i.e., tracker 52B), or any one or more links 18 of the robotic manipulator 14. The trackers 52, 54, 56, PT may be fixed to their respective components in any suitable manner.

Any one or more of the trackers may include active markers 58. The active markers 58 may include light emitting diodes (LEDs). Alternatively, the trackers 52, 54, 56 may have passive markers, such as reflectors, which reflect light emitted from the camera unit 46. Other suitable markers not specifically described herein may be utilized.

The localizer 44 tracks the trackers 52, 54, 56 to determine a state of each of the trackers 52, 54, 56, which correspond respectively to the state of the object respectively attached thereto. The localizer 44 provides the state of the trackers 52, 54, 56 to the navigation computer 36. In one example, the navigation computer 36 determines and communicates the state the trackers 52, 54, 56 to the manipulator computer 26. As used herein, the state of an object includes, but is not limited to, data that defines the position and/or orientation of the tracked object or equivalents/derivatives of the position and/or orientation. For example, the state may be a pose of the object, and may include linear data, and/or angular velocity data, and the like.

Although one example of the navigation system 32 is shown in the Figures, the navigation system 32 may have any other suitable configuration for tracking the robotic manipulator 14 and the patient 12. In one example, the navigation system 32 and/or localizer 44 are ultrasound-based. In another example, the navigation system 32 and/or localizer 44 are radio frequency (RF)-based.

The navigation system 32 and/or localizer 44 may have any other suitable components or structure not specifically recited herein. Furthermore, any of the techniques, methods, and/or components described above with respect to the camera-based navigation system 32 shown throughout the Figures may be implemented or provided for any of the other examples of the navigation system 32 described herein. For example, the navigation system 32 may utilize solely inertial tracking or any combination of tracking techniques.

The controller 30 further includes software modules. The software modules may be part of a computer program or programs that operate on the manipulator computer 26, navigation computer 36, or a combination thereof, to process data to assist with control of the system 10. The software modules include instructions stored in memory on the manipulator computer 26, navigation computer 36, or a combination thereof, to be executed by one or more processors of the computers 26, 36. Additionally, software modules for prompting and/or communicating with the operator may form part of the program or programs and may include instructions stored in memory on the manipulator computer 26, navigation computer 36, or a combination thereof. The operator interacts with the first and second input devices 40, 42 and the one or more displays 38 to communicate with the software modules. The user interface software may run on a separate device from the manipulator computer 26 and navigation computer 36.

The controller 30 includes a manipulator controller 60 for processing data to direct motion of the robotic manipulator 14. In one example, as shown in FIG. 1, the manipulator controller is implemented on the manipulator computer 26. The manipulator controller 60 may receive and process data from a single source or multiple sources. The controller 30 further includes a navigation controller 62 for communicating the state data relating to the femur F, tibia T, and robotic manipulator 14 to the manipulator controller 60. The manipulator controller 60 receives and processes the state data provided by the navigation controller 62 to direct movement of the robotic manipulator 14. In one example, as shown in FIG. 1, the navigation controller 62 is implemented on the navigation computer 36. The manipulator controller 60 or navigation controller 62 may also communicate states of the patient 12 and robotic manipulator 14 to the operator by displaying an image of the femur F and/or tibia T and the robotic manipulator 14 on the one or more displays 38. The manipulator computer 26 or navigation computer 36 may also command display of instructions or request information using the display 38 to interact with the operator and for directing the robotic manipulator 14.

The controller 30 includes a boundary generator 66. The boundary generator 66 is a software module that may be implemented on the manipulator controller 60. Alternatively, the boundary generator 66 may be implemented on other components, such as the navigation controller 62. The boundary generator 66 generates virtual boundaries for constraining the tool 20.

Such virtual boundaries may also be referred to as virtual meshes, virtual constraints, or the like. The virtual boundaries may be defined with respect to a 3-D bone model registered to the one or more patient trackers 54, 56 such that the virtual boundaries are fixed relative to the bone model. The state of the tool 20 is tracked relative to the virtual boundaries. In one example, the state of the TCP is measured relative to the virtual boundaries for purposes of determining when and where haptic feedback force is applied to the robotic manipulator 14, or more specifically, the tool 20.

A tool path generator 69 is another software module run by the controller 30, and more specifically, the manipulator controller 60. The tool path generator 69 generates a path 100 for the tool 20 to traverse, such as for removing sections of the anatomy to receive an implant. One exemplary system and method for generating the tool path 100 is explained in U.S. Pat. No. 9,119,655, entitled, “Surgical Manipulator Capable of Controlling a Surgical Instrument in Multiple Modes,” the disclosure of which is hereby incorporated by reference. In some examples, the virtual boundaries and/or tool paths 100 may be generated offline rather than on the manipulator computer 26 or navigation computer 36. Thereafter, the virtual boundaries and/or tool paths 100 may be utilized at runtime by the manipulator controller 60.

### II. Surgical Driving System

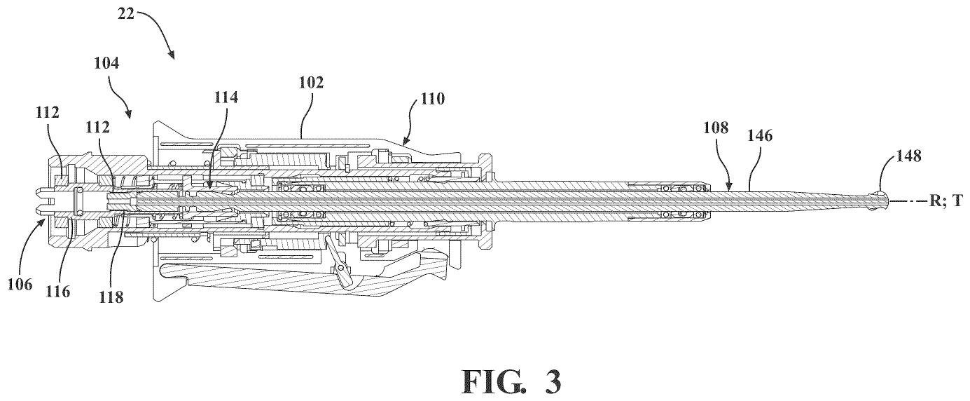

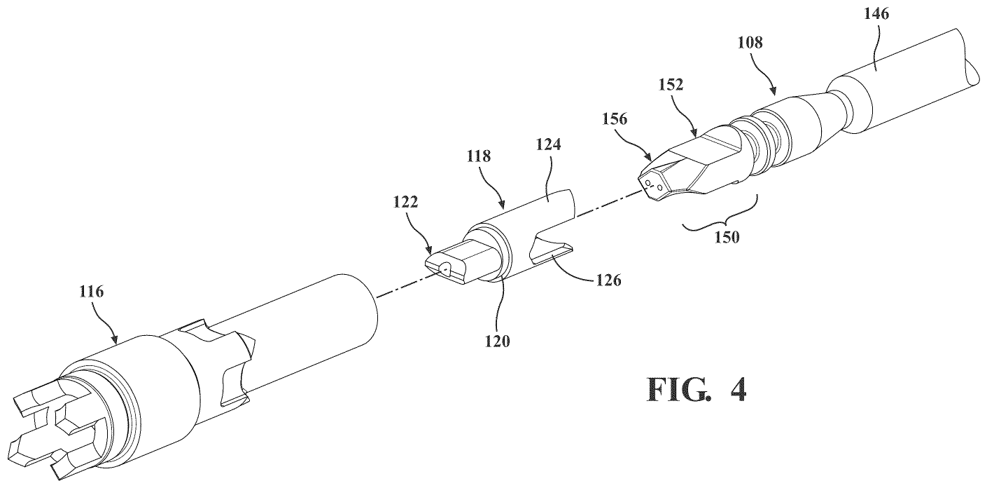

Referring to the Figures, wherein like numerals indicate like or corresponding parts throughout the several views, a surgical driving system 104 is generally shown in FIG. 3. The surgical driving system 104 comprises a drive assembly 106 and a cutting accessory 108. As will be described below, the surgical driving system 104 may be utilized with the system 10, and more specifically, the surgical driving system 104 may be utilized for orienting the cutting accessory 108 to the end effector 22. The drive assembly 106 receives torque from a motor (not shown) of the end effector 22 and transfers the torque to the cutting accessory 108 to operate the cutting accessory 108. It is contemplated that the surgical driving system 104 may be utilized for other types of surgical components other than the cutting accessory 108 and the end effector 22. For instance, the cutting accessory 108 may be realized as another type of energy applicator that is configured to receive torque from a motor.

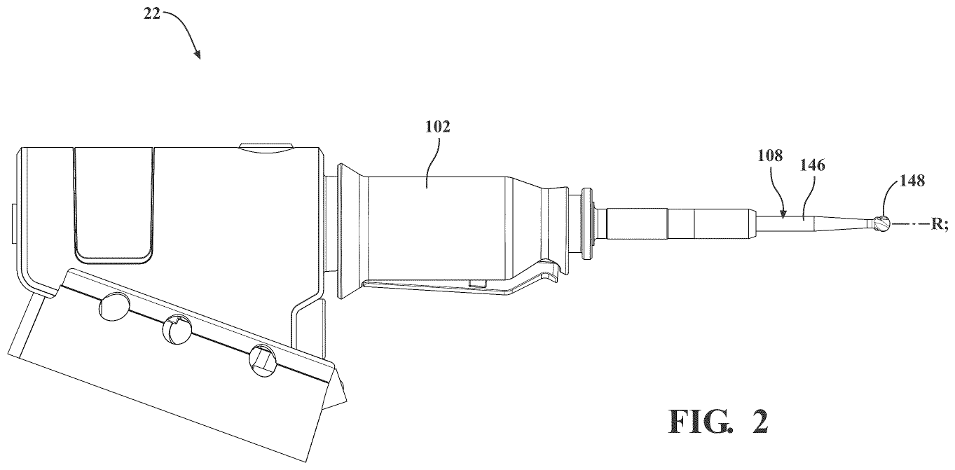

As shown in FIGS. 2 and 3, the end effector 22 including the surgical driving system 104 is shown. The end effector 22 may also include a housing 110 with bearings 112 to support rotation of the drive assembly 106 and the cutting accessory 108. The end effector 22 may also include a coupling system 114 to removably secure the cutting accessory 108 to the end effector 22. Examples of suitable coupling systems may be found in the aforementioned U.S. Patent Publication No. 2022/0087754A1, the disclosure of which is hereby incorporated by reference in its entirety. The coupling system 114 may be used to secure the cutting accessory 108 after the cutting accessory 108 has been properly oriented to the drive assembly 106 and the cutting accessory 108 is engaged with the drive assembly 106. Features for orienting the cutting accessory 108 relative to the drive assembly 106 are described in greater detail further below. The end effector 22 may further include the motor to generate torque. The drive assembly 106 is configured to be coupled to the motor (e.g., a motor shaft of the motor) to receive torque from the motor to operate the cutting accessory 108 when the cutting accessory 108 is engaged with the drive assembly 106.

As shown in FIG. 3, the coupling system 114 may be disposed distal the drive assembly 106. The cutting accessory 108 may be introduced through the coupling system 114 before approaching the drive assembly 106. A user may attempt to orient the cutting accessory 108 relative to the drive assembly 106 so that the drive assembly 106 may engage the cutting accessory 108 to permit torque transfer between the drive assembly 106 and the cutting accessory 108. As may be appreciated from FIG. 3, the user may not have visibility of a proximal portion of the cutting accessory 108 and/or the drive assembly 106. Thus, ensuring proper orientation between the cutting accessory 108 and the drive assembly 106 by sight alone may be difficult. The cutting accessory 108 and the drive assembly 106 may include alignment features, described in greater detail below, to facilitate proper orientation between the drive assembly 106 and the cutting accessory 108 sufficient to engage the drive assembly 106 with the cutting accessory 108.

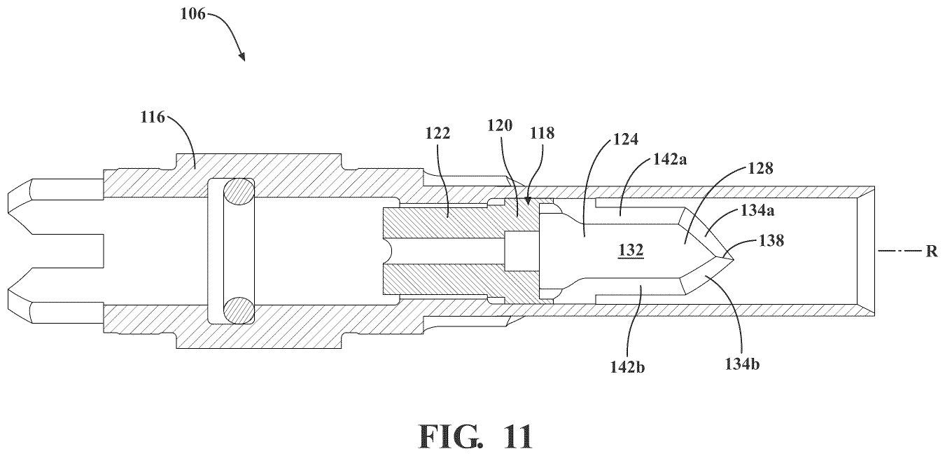

As shown in FIGS. 10 and 11, the drive assembly 106 may include a drive member 116 and a driver 118. The drive member 116 may be coupled to an input shaft such as a motor armature or another shaft configured to receive torque from the motor. In some configurations, a gear assembly or another form of transmission may be disposed between the motor and the drive member 116 to adjust a rotational speed of the drive member 116 relative to the motor. The drive assembly 106 also includes a driver 118 that may be coupled to the drive member 116 to receive torque from the motor through the drive member 116 for engaging the cutting accessory 108. The driver 118 may be coupled to the drive member 116 such that the driver 118 and the drive member 116 rotate in unison about the rotational axis R. In some configurations, the driver 118 and the drive member 116 are monolithic. In other words, the driver 118 and the drive member 116 may comprise a single rigid body. The drive member 116 and/or the driver 118 may include a body 120 having a proximal portion 122 configured to receive torque from the motor and rotate about the axis R. In some configurations, the proximal portion 122 of the body 120 of the driver 118 may include flats configured to receive torque from the motor.

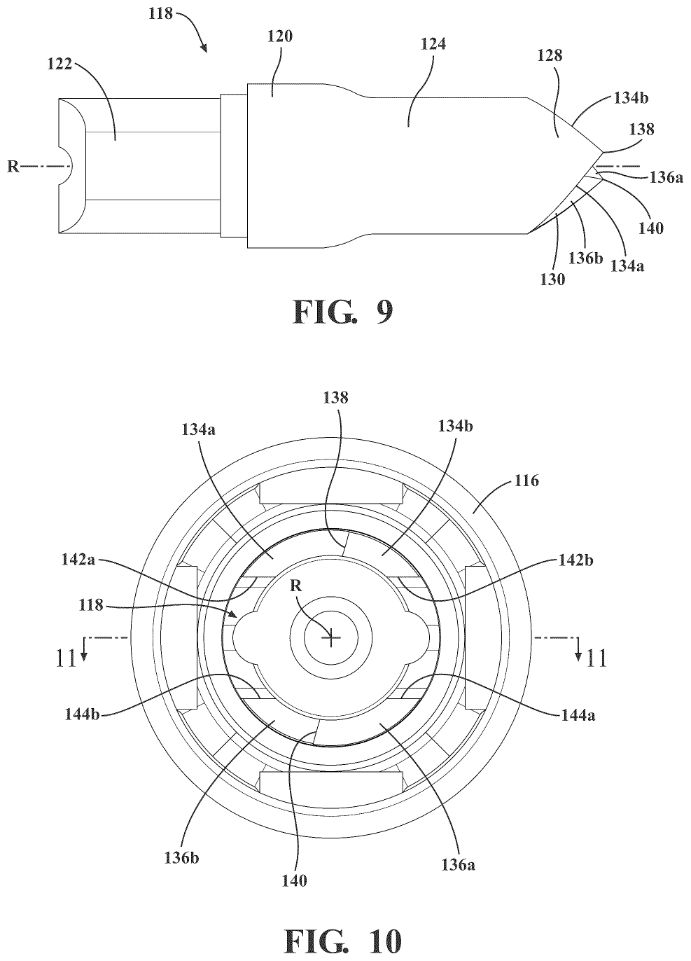

As shown in FIGS. 8-11, the driver 118 may include first and second arms 124, 126 projecting distally from the body 120. The first and second arms 124, 126 are spaced apart from one another. The first arm 124 may include a first alignment tip 128. The second arm 126 may include a second alignment tip 130. In some configurations, a single alignment tip 128, 130 on one of the first or second arms 124, 126 is employed. The first and second arms 124, 126 may collectively define a channel 132 between the first and second arms 124, 126 for receiving the cutting accessory 108 when the cutting accessory 108 is introduced to the driver 118. The alignment tips 128, 130 may each include two alignment surfaces 134a, 134b, 136a, 136b facing away from each other. The two alignment surfaces 134a, 134b, 136a, 136b of each alignment tip 128, 130 are arranged for selectively imparting relative rotation of the cutting accessory 108 relative the driver 118 during engagement with the cutting accessory 108. In some configurations, the driver 118 may be temporarily fixed from rotation (e.g., with a motor brake) and the cutting accessory 108 may be configured to rotate to the correct orientation to engage the driver 118 such that torque transmission is possible. For instance, the user may allow the cutting accessory 108 to rotate in their hand in response to the cutting accessory 108 abutting one or more of the alignment surfaces 134a, 134b, 136a, 136b while urging the cutting accessory 108 proximally relative to the driver 118 to allow the cutting accessory 108 to engage the driver 118. In other configurations, the user may introduce the cutting accessory 108 such that the cutting accessory 108 is fixed from rotation (e.g., by firmly grasping the cutting accessory 108 in the user's hand) and the at least one of the alignment surfaces 134a, 134b, 136a, 136b may engage the cutting accessory 108 so that the driver 118 rotates relative to the cutting accessory 108 to the correct orientation to engage the driver 118 such that torque transmission is possible.

The second alignment tip 130 may be offset from the first alignment tip 128 such that the first alignment tip 128 is located on one side of a reference plane that bisects the axis R and the body 120 and the second alignment tip 130 is located on an opposing side of the reference plane. Said differently, the reference plane may extend through the axis R such that the axis R is coincident with the reference plane. In some configurations, one or more of the alignment surfaces 134a, 134b, 136a, 136b are helically contoured. In other configurations, one or more of the alignment surfaces 134a, 134b, 136a, 136b are planar. The offset arrangement of the first and second alignment tips 128, 130 may mitigate jamming that may otherwise occur when the cutting accessory 108 is introduced to the driver 118.

As shown in FIG. 10, the alignment surfaces 134a, 134b, 136a, 136b of each alignment tip 128, 130 may share one distal edge 138, 140 to collectively form the respective alignment tip 128, 130. The first distal edge 138 of the first alignment tip 128 may extend along a first reference line. The second distal edge 140 of the second alignment tip 130 may extend along a second reference line. The first and second reference lines may be parallel when viewed from the distal end (as viewed in FIG. 10). The first and second reference lines may not intersect the axis R. This arrangement of the distal edges 138, 140 may mitigate jamming that may otherwise occur when the cutting accessory 108 is introduced to the driver 118.

The first and second arms 124, 126 may each include at least one drive surface 142a, 142b, 144a, 144b disposed proximal to the respective alignment surfaces 134a, 134b, 136a, 136b so that as the cutting accessory 108 is loaded into the end effector 22, axial manipulation of the cutting accessory 108 against the alignment surfaces 134a, 134b, 136a, 136b of the driver 118 causes the cutting accessory 108 to rotate so that corresponding drive surfaces of the cutting accessory 108 are brough into an orientation and axial position sufficient to engage the drive surfaces 142a, 142b, 144a, 144b of the driver 118. The drive surfaces 142a, 142b, 144a, 144b of the driver 118 may be located within the channel 132. The alignment surfaces 134a, 134b, 136a, 136b of each arm 124, 126 may be arranged so that one of the alignment surfaces 134a, 134b, 136a, 136b urges the cutting accessory 108 in one direction and the other alignment surface 134a, 134b, 136a, 136b urges the cutting accessory 108 in the opposite direction. The direction the cutting accessory 108 rotates may be determined by which one of the alignment surfaces 134a, 134b, 136a, 136b abuts the cutting accessory 108. Each drive surface 142a, 142b, 144a, 144b of the driver 118 may extend from a proximal edge of one of the alignment surfaces 134a, 134b, 136a, 136b such that the proximal edge of the alignment surface 134a, 134b, 136a, 136b is also the distal edge of the respective drive surface 142a, 142b, 144a, 144b. Each of the first and second arms may include two drive surfaces 142a, 142b, 144a, 144b. The two drive surfaces 142a, 142b, 144a, 144b of each arm 124, 126 may be flat and coplanar with one another. The two drive surfaces 142a, 142b of the first arm 124 may be parallel with and arranged to face the two drive surfaces 144a, 144b of the second arm 126.

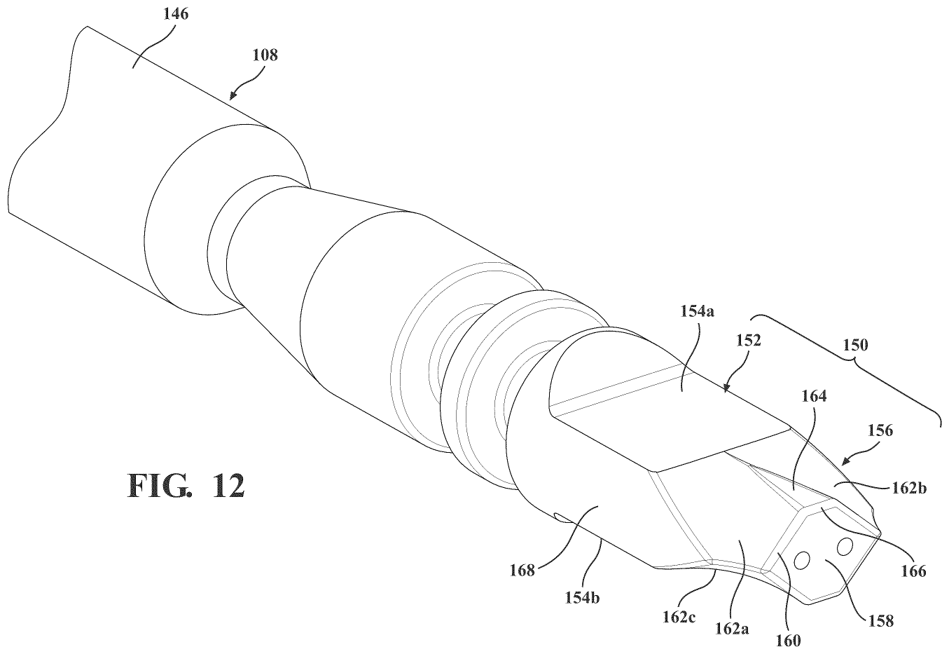

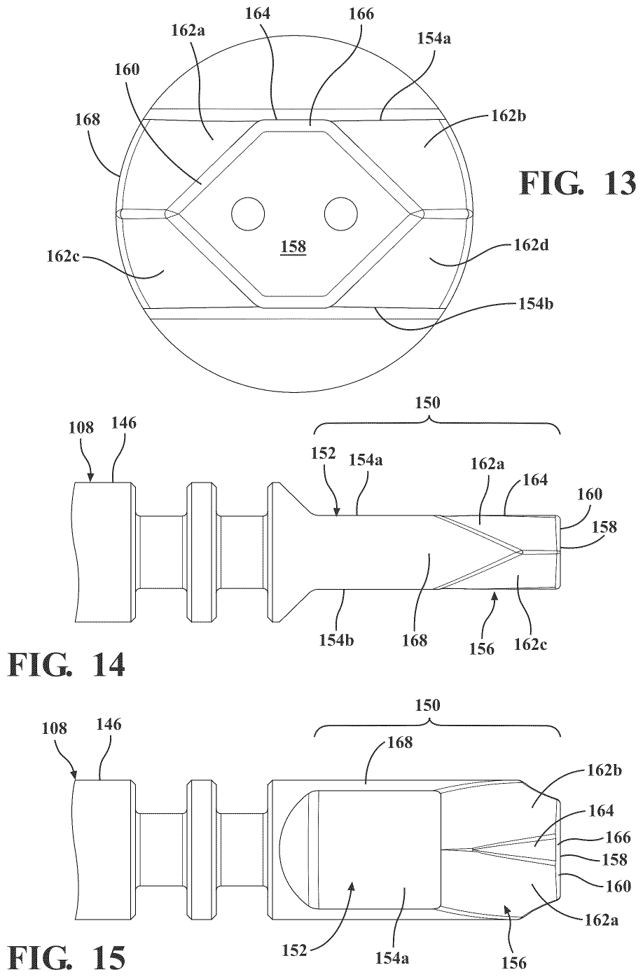

As shown in FIGS. 3 and 12-15, the cutting accessory 108 may include a tool shaft 146 extending along a tool axis T and being rotatable about the tool axis T. When the cutting accessory 108 is engaged with the driver 118, the tool axis T is collinear with the rotational axis R. The cutting accessory 108 includes a distal portion 148 coupled to the tool shaft 146. The distal portion 148 may have a cutting tip such as a bur head, a drill bit, or another configuration for cutting tissue. The cutting accessory 108 also includes a proximal portion 150 coupled to the tool shaft 146 and configured to engage the driver 118 of the drive assembly 106 so that the driver 118 may transfer torque to the cutting accessory 108. The proximal portion 150 includes a drive section 152 having one or more flats 154a, 154b configured to engage the drive surfaces 142a, 142b, 144a, 144b of the driver 118 to receive torque from the driver 118. In some configurations, the drive section 152 may include a first flat 154a and a second flat 154b being opposite and parallel to the first flat 152a.

The proximal portion 150 also includes an alignment section 156 distinct from and proximal to the drive section 152. The alignment section 156 is configured to orient the drive section 152 to the drive surfaces 142a, 142b, 144a, 144b of the driver 118 so that the flats 154a, 154b of the drive section 152 may be in contact with the drive surfaces 142a, 142b, 144a, 144b of the driver 118. The alignment section 156 includes a proximal end 158 having at least one edge 160. The alignment section 156 also includes an alignment surface 162a extending distally from the at least one edge 160 of the proximal end 158 toward the drive section 152. In some configurations, the alignment surface 162a may be non-planar. In some configurations, the alignment surface 162a is helically twisted between the at least one edge 160 of the proximal end 158 of the alignment section 156 and the drive section 152. The alignment surface 162a may extend to a proximal edge of the drive section 152. The alignment section 156 may be formed such that a cross-sectional area of the alignment section 156 may increase along the tool axis T from the proximal end 158 to the proximal edge of the drive section 152. The alignment surface 162a may be configured to ramp upward (e.g., outwardly from the axis T) from the proximal end 158 of the alignment section 156 to the proximal edge of the drive section 152. The at least one edge 160 of the alignment section 156 may be coincident to a reference edge plane that extends parallel to the tool axis T. The alignment surface 162a may be twisted such that the alignment surface 162a deviates from the reference edge plane. In other words, the alignment surface 162a may extend distally from the proximal edge 160 with a non-planar surface.

The proximal end 158 may have a proximal surface. The proximal surface may be planar and perpendicular to the tool axis T. In other configurations, the proximal surface may be non-planar. The proximal surface may have a polygonal cross-section with at least one edge of the polygonal cross-section forming the at least one edge 160 of the proximal end 158 of the alignment section 156. In some configurations, the proximal surface may include a hexagonal cross-section with at least one edge of the hexagonal cross-section forming the at least one edge 160 of the proximal end 158 of the alignment section 156. In other configurations, the proximal end 158 may have a circular or oval cross-section with a circumferential edge of the circular or oval cross-section forming the at least one edge 160 of the proximal end 158 of the alignment section 156.

The alignment surface 160a may be defined as a first alignment surface 160a and the alignment section 156 may include a second alignment surface 156b that is symmetrical to the first alignment surface 160a about a first reference plane that bisects the tool axis T such that the reference plane includes the tool axis T. The alignment section 156 may also include a third alignment surface 160c symmetrical to the first alignment surface 160a about a second reference plane that bisects the tool axis T and is perpendicular to the first reference plane. The alignment section 156 may also include a fourth alignment surface 160d symmetrical to the second alignment surface 160b about the second reference plane. The alignment section 156 may include a planar surface 164 extending distally from at least one other edge 166 of the proximal end 158 of the alignment section 156. The planar surface 164 may extend toward the drive section 152. The planar surface 164 may be configured to separate the first and second alignment surfaces 160a, 160b. The alignments surfaces 160a, 160b, 160c, 160d may be cut into the proximal portion 150 of the cutting accessory 108 such that outer alignment edges are formed between each of the alignment surfaces 160a, 160b, 160c, 160d and an outer surface 168 of the proximal portion 150 of the cutting accessory 108.

In one exemplary configuration, the user would grasp the cutting accessory 108 and prepare to couple the cutting accessory 108 to the end effector 22. The user introduces the cutting accessory 108 into a distal opening of the end effector 22 and axially displaces the cutting accessory 108 until the cutting accessory 108 reaches the driver 118. If the flats 154a, 154b of the drive section 152 are oriented for engagement with the drive surfaces 142a, 142b, 144a, 144b of the driver 118 (e.g., the flats 154a, 154b are parallel with the drive surfaces 142a, 142b, 144a, 144b), then the user may continue to axially displace the cutting accessory 108 relative to the driver 118 such that the cutting accessory 108 may be fully loaded without rotating either the driver 118 or the cutting accessory 108 so that the driver 118 may transfer torque to the cutting accessory 108.

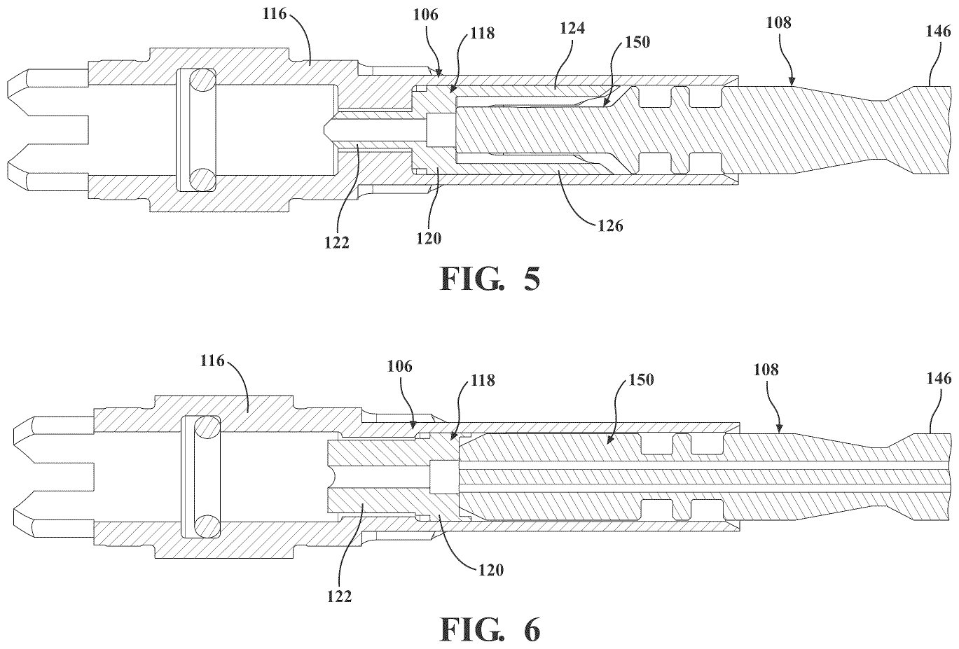

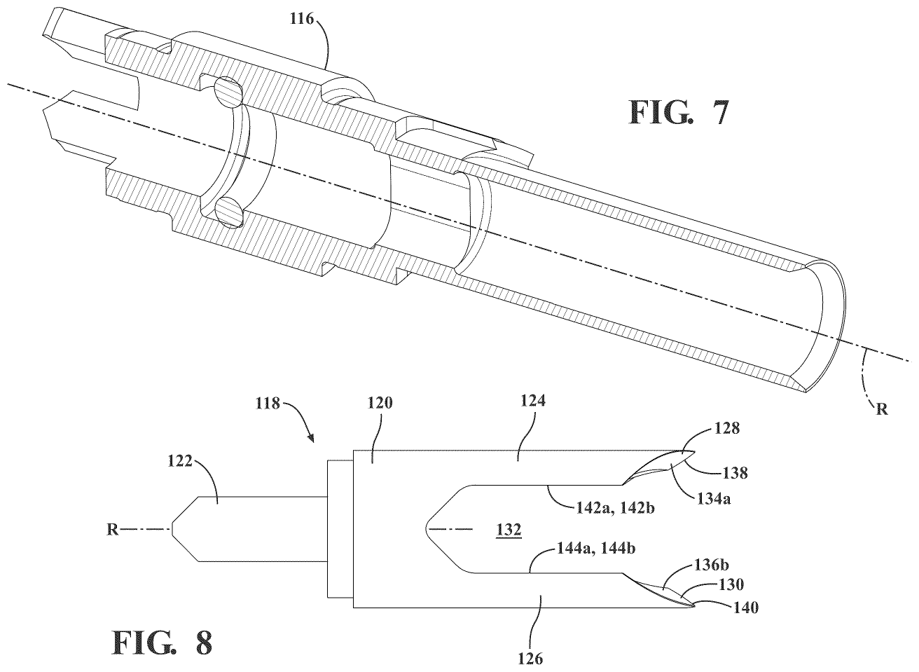

If the flats 154a, 154b of the drive section 152 are not oriented for engagement with the drive surfaces 142a, 142b, 144a, 144b of the driver 118, then the alignment surfaces 160a, 160b, 160c, 160d of the cutting accessory 108 or one of the edges formed in part by the alignment surfaces 160a, 160b, 160c, 160d of the cutting accessory 108 abut the alignment surfaces 134a, 134b, 136a, 136b of the alignment tips 128, 130 of the driver 118. Continued axial displacement of the cutting accessory 108 within the end effector 22 relative to the driver 118 will cause relative rotation between the cutting accessory 108 and the driver 118 by virtue of the abutting alignment surfaces 134a, 134b, 136a, 136b, 160a, 160b, 160c, 160d until the flats 154a, 154b of the drive section 152 of the cutting accessory 108 are oriented for engagement with the drive surfaces 142a, 142b, 144a, 144b of the driver 118 (as shown in FIGS. 5 and 6).

Several configurations have been discussed in the foregoing description. However, the configurations discussed herein are not intended to be exhaustive or limit the invention to any particular form. The terminology which has been used is intended to be in the nature of words of description rather than of limitation.

What is claimed is:

1. A cutting accessory for a surgical cutting tool, the cutting accessory comprising:
a tool shaft comprising an axis and being rotatable about the axis;
a distal portion coupled to the tool shaft and having a cutting tip; and
a proximal portion coupled to the tool shaft and configured to engage a driver of the surgical cutting tool, the proximal portion comprising:
a drive section having one or more flats configured to engage drive surfaces of the driver to receive torque from the driver, and
an alignment section distinct from and proximal to the drive section, the alignment section configured to orient the drive section to the drive surfaces of the driver, and the alignment section comprising,
a proximal end comprising at least one edge, and
an alignment surface extending distally from the at least one edge of the proximal end toward the drive section, and wherein the alignment surface is twisted relative to the axis between the at least one edge of the proximal end and the drive section.

2. The cutting accessory of claim 1, wherein the alignment surface is helically twisted between the at least one edge of the proximal end and the drive section.

3. The cutting accessory of claim 1, wherein the alignment surface extends to an edge of the drive section.

4. The cutting accessory of claim 1, wherein a cross-sectional area of the alignment section is configured to increase along the axis from the proximal end to an edge of the drive section.

5. The cutting accessory of claim 1, wherein the alignment surface is configured to ramp upward from the proximal end to an edge of the drive section.

6. The cutting accessory of claim 1, wherein:
the at least one edge is coincident to a reference edge plane that extends parallel to the axis; and
the alignment surface is twisted such that the alignment surface deviates from the reference edge plane.

7. The cutting accessory of claim 1, wherein:
the proximal end comprises a polygonal cross-section and at least one edge of the polygonal cross-section forms the at least one edge;
the proximal end comprises a hexagonal cross-section and at least one edge of the hexagonal cross-section forms the at least one edge;
the proximal end comprises a circular cross-section, and at least a portion of a circumferential edge of the circular cross-section forms the at least one edge; or
the proximal end comprises an oval cross-section, and at least a portion of a circumferential edge of the oval cross-section forms the at least one edge.

8. The cutting accessory of claim 1, wherein the proximal end is planar and perpendicular to the axis.

9. The cutting accessory of claim 1, wherein the proximal end is non-planar.

10. The cutting accessory of claim 1, wherein the drive section comprises a first flat and a second flat being opposite and parallel to the first flat.

11. The cutting accessory of claim 1, wherein the alignment surface is further defined as a first alignment surface, and wherein the alignment section comprises:
a second alignment surface symmetrical to the first alignment surface about a first reference plane that bisects the axis.

12. The cutting accessory of claim 11, wherein the alignment section comprises:
a third alignment surface symmetrical to the first alignment surface about a second reference plane that bisects the axis and is perpendicular to the first reference plane; and
a fourth alignment surface symmetrical to the second alignment surface about the second reference plane.

13. The cutting accessory of claim 11, wherein the alignment section comprises a planar surface extending distally from at least one other edge of the proximal end toward the drive section, the planar surface configured to separate the first and second alignment surfaces.

14. A driver of a surgical tool for engaging a cutting accessory, the driver comprising:
a body having a proximal portion configured to receive torque from a motor and rotate about an axis;
a first arm projecting distally from the body, the first arm having a first alignment tip; and
a second arm projecting distally from the body and being spaced from the first arm, the second arm having a second alignment tip;
the first and second arms collectively defining a channel between the first and second arms for at least partially receiving the cutting accessory;
wherein each of the alignment tips include two alignment surfaces facing away from each other, the two alignment surfaces being arranged for selectively imparting rotation of the cutting accessory during engagement with the cutting accessory; and
wherein the second alignment tip is offset from the first alignment tip such that the first alignment tip is located on one side of a reference plane that bisects the axis and the body and the second alignment tip is located on an opposing side of the reference plane.

15. The driver of claim 14, wherein each alignment surface is helically contoured.

16. The driver of claim 14, wherein, for each arm, the two alignment surfaces share one distal edge to form the alignment tip.

17. The driver of claim 14, wherein each of the first and second arms comprise at least one drive surface disposed proximal to the alignment surfaces and being located within the channel, the at least one drive surface being configured to engage a corresponding drive surface of the cutting accessory.

18. The driver of claim 17, wherein each alignment surface ramps up or down to at a distal edge of the at least one drive surface.

19. The driver of claim 17, wherein each drive surface extends from a proximal edge of one of the alignment surfaces.

20. The driver of claim 17, wherein:
each of the first and second arms comprise two drive surfaces;
the two drive surfaces of each arm are flat and coplanar with one another; and
the two drive surfaces of the first arm are parallel with and facing the two drive surfaces of the second arm.

21. The driver of claim 14, wherein the proximal portion of the body comprises flats configured to receive torque from the motor.

22. A surgical tool comprising:
a motor; and
a driving system configured to transmit torque received from the motor, the driving system comprising:
a driver configured to receive torque from the motor, the driver comprising:
a body having a proximal portion configured to receive torque from the motor and rotate about an axis;
a first arm projecting distally from the body, the first arm having a first alignment tip; and
a second arm projecting distally from the body and being spaced from the first arm, the second arm having a second alignment tip;
the first and second arms collectively defining a channel between the first and second arms;
each of the alignment tips include two alignment surfaces facing away from each other, wherein the second alignment tip is offset from the first alignment tip such that the first alignment tip is located on one side of a reference plane that bisects the axis and the body and the second alignment tip is located on an opposing side of the reference plane; and
a cutting accessory configured to be coupled to the driver, the cutting accessory comprising:
a tool shaft comprising an axis and being rotatable about the axis;
a distal portion coupled to the tool shaft and having a cutting tip; and
a proximal portion coupled to the tool shaft and configured to be received within the channel to engage the driver, the proximal portion comprising:
a drive section having one or more flats configured to engage the first and second arms of the driver to receive torque from the driver, and
an alignment section distinct from and proximal to the drive section, the alignment section configured to engage the alignment tips of the driver to orient the drive section relative to the arms.
